(19) United States
(12) Patent Application Publication
(10) Pub. No.: US 2008/0299165 A1
(43) Pub. Date: Dec. 4, 2008

(54) Compositions for Treatment of Diseases of the Nail Unit

(76) Inventors: Jing-song Tao, Vancouver (CA); Ronald Erwin Boch, North Vancouver (CA); Alain H. Curaudeau, West Vancouver (CA); Peter Lutwyche, Vancouver (CA); Agnes How-Ching Chan, Coquitlam (CA)

Correspondence Address: SCHWEGMAN, LUNDBERG & WOESSNER, P.A., P.O. BOX 2938, MINNEAPOLIS, MN, 55402, US

(21) Appl. No.: 11/884,321

(22) PCT Filed: Feb. 17, 2006
(86) PCT No.: PCT/CA2006/000240

§ 371 Date: Apr. 24, 2008

(30) Foreign Application Priority Data
Feb. 18, 2005 (CA) 2,498,623

(51) Int. Cl. A61K 9/00; A61K 47/30; A61K 31/13; A61K 31/4353; A61K 38/13; A61P 31/10; A61P 31/12

(52) U.S. Cl. 424/422; 514/772.3; 514/655; 514/291; 514/11

(57) ABSTRACT

A flowable composition suitable for use as a controlled release implant, the composition comprising a biodegradable thermoplastic polyester that is at least substantially insoluble in aqueous medium or body fluid; a biocompatible polar aprotic solvent selected from the group consisting of an amide, an ester, a carbonate, a ketone, an ether, and a sulfonyl; wherein the biocompatible polar aprotic solvent is miscible to dispersible in aqueous medium or body fluid, and antifungal active.

This application claims priority from Canadian Patent Application No. 2,498,623, filed Feb. 18, 2005, the entire contents of which is incorporated herein by reference.

### FIELD OF THE INVENTION

This invention relates to compositions, devices, kits, methods, uses and systems for treating diseases of the nail unit.

### BACKGROUND OF THE INVENTION

Diseases of the nail unit such as those caused by fungal, bacterial or viral infection, or by autoimmune or inflammatory diseases, or hyperproliferative disorders, are often common but difficult to treat.

The nail unit is generally composed of the nail plate, the nail bed, the hyponychium, the nail matrix, the nail folds, the cuticle, the anchoring portion of the nail bed, and the distal phalangeal bones. The nail plate is the largest component of the nail unit. The nail matrix gives rise to the nail plate. Delivery of active ingredients to components of the nail unit can be challenging.

Diseases of the nail unit relating to antimicrobial infections, include but are not limited to, onychomycosis. Onychomycosis is a common dermatological condition that results from a fungal infection of the nail apparatus. The condition has a variety of clinical classifications and outcomes but often results in undesirable changes to the condition of the nail which may cause discomfort or pain to the sufferer. There are several treatments currently indicated for onychomycosis in the United States. One topical treatment is ciclopirox nail lacquer which, in two pivotal clinical trials, reported a mycologic cure rate of between 29% and 36% for mild to moderate toenail onychomycosis. The main systemic treatment for onychomycosis is terbinafine although griseofulvin and itaconazole are also approved for the condition. Terbinafine has a reported efficacy of 38% to 70% but can have unwanted side-effects, such as lower toxicity. Treatment of onychomycosis can also include surgical or chemical removal of the nail plate.

Another common infection of the nail unit is caused by vital wart infection of the nail fold and nail bed, commonly referred to as periungual vital warts. Treatment for this condition includes laser surgery.

Yet another example of a microbial infection to the nail unit is paronychia, which is an inflammation of the nail folds which can often result from injury to the proximal nail fold. Secondary infection by bacteria or fungus can cause painful swelling of the nail fold. At present, treatments for paronychia include the drainage of pus from the infected nail fold and treatment with oral antibiotics. Topical antifungal and antiseptic lotions are also used to treat paronychia.

Diseases of the nail unit relating to autoimmune disorders include, but are not limited to, nail psoriasis. Nail psoriasis is common in subjects with active psoriasis. Psotiatic changes in nails range from mild to severe, generally reflecting the extent of psoriatic involvement of the nail plate, nail matrix, nail bed, and skin at the base of the nail. Psoriasis of the nail is currently difficult to treat. Injections of steroids into the nail bed or matrix area have been used with varying results, and ongoing injections in the nail during the course of treatment can be painful to the patient or perceived to be painful by the patient

Diseases of the nail unit relating to a disregulation of cellular proliferation include, but are not limited to, nail melanoma. In the early stages, treatment consists of surgically removing the melanoma. More advanced melanoma may require amputation of the affected finger or toe along with possible removal of the regional lymph nodes and chemotherapy.

Overall, treatment of diseases of the nail unit remains challenging. While systemic oral drug treatments are currently known for the treatment of various diseases of the nail unit, such treatments generally can have the disadvantage of having poor efficacy and displaying side effects associated with systemic administration.

Because of ease in administration and comfort, topical treatments that treat nail diseases by imparting an active agent to the nail plate, and through the nail plate to the nail bed are generally preferable to oral administration or to surgery. However, delivery of drugs through the nail plate has proven to be difficult due to the thickness and relative impermeability of the nail plate. Several prior art topical nail therapies are known to be generally ineffective for treating many conditions of the nail unit, particularly those of the nail bed given the difficulty in delivering active ingredients through the nail plate to the nail bed. Topical applications also often require lengthy periods of repeated administration, which can result in poor patient compliance.

There remains a continuing need for the development of effective means for the treatment of diseases of the nail unit, particularly diseases that call for delivery of active ingredient to the nail unit for sustained periods of time.

### SUMMARY OF THE INVENTION

The present disclosure relates to methods, devices, compositions, and kits for treating diseases of the nail bed.

Therefore, in one aspect of the present invention is provided a flowable composition suitable for periungually or subungually forming in situ a controlled release biodegradable implant system, the composition comprising: (a) a biodegradable pharmaceutically acceptable thermoplastic polymer that is at least substantially insoluble in aqueous medium or body fluid; (b) a pharmaceutically acceptable biocompatible solvent that is water soluble; and (c) a therapeutically effective amount of an active ingredient, wherein the thermoplastic polymer and biocompatible solvent are present in concentrations effective to form the implant in situ. In one embodiment, the biodegradable pharmaceutically acceptable thermoplastic polymer comprises a biodegradable thermoplastic polyester that is at least substantially insoluble in aqueous medium or body fluid. In another embodiment of the present invention, the pharmaceutically acceptable biocompatible solvent comprises a biocompatible polar aprotic solvent selected from the group consisting of an amide, an ester, a carbonate, a ketone, an ether, and a sulfonyl; wherein the biocompatible polar aprotic solvent is miscible to dispersible in aqueous medium or body fluid. In another embodiment, the active ingredient comprises an antimicrobial agent, an antibiotic agent, an antifungal agent, an immunosuppressive agent or a anti-hyperproliferative agent.

In another aspect of the present invention is provided depositing a therapeutically effective composition containing an active agent periungually or subungually in a subject affected by a disease of the nail unit. The present compositions include those which deliver the active agents in a controlled-release manner over a period of time. The present devices include implants for insertion for periungually or subungually that comprise an active agent. The present kits include those comprising a composition or implant comprising an active agent and instructions for inserting the composition under the nail of a subject affected by a microbial infection.

In one embodiment of the invention is provided depositing a composition periungually or subungually in a subject affected by a microbial infection. The present compositions include those which deliver antimicrobial agents in a controlled-release manner over a sustained period of time. The present devices include implants for insertion for periungually or subungually that comprise an antimicrobial agent. The present kits include those comprising a composition or device comprising an antimicrobial agent and instructions for inserting the composition periungually or subungually in a subject affected by a microbial infection. In one aspect of the invention, the microbial infection comprises onychomycosis and the antifungal is selected from ciclopitox, naftifine, griseofulvin, itraconazole, terbinafine, ketoconazole, fluconazole and the like, including suitable salts and derivatives thereof, as well as rapamycin and FK506, and mixtures thereof. In another aspect of the present invention, the microbial infection comprises a bacterial infection and the antibacterial agent is selected from penicillins, cephalosporins, vancomycins, bacitracin, polymycins, tetracyclines, erythromycin, streptomycin and the like. In yet another aspect of the present invention, the microbial infection comprises a vital infection and the antiviral is selected from bleomycin, acyclovir, valcyclovir, famcyclovir, and the like.

In another aspect of the invention is provided a composition inserted periungually or subungually in a subject affected by an autoimmune disease. The present biodegradable implants include those which deliver immunosuppressive agents in a controlled-release manner over a period of time. The present devices include implants for insertion periungually or subungually that comprise an immunosuppressive agent. The present kits include those comprising a composition or device comprising an immunosuppressive agent and instructions for inserting the composition periungually or subungually in a subject affected by an autoimmune disease. In one embodiment of the present invention, the autoimmune disorder is nail psoriasis and the immunosuppressive agent is rapamycin or cyclosporine, or the like.

In another aspect of the invention is provided depositing an antihyperproliferative agent containing composition periungually or subungually in a subject affected by a hyperproliferative disease. The present biodegradable implants include those which deliver antihyperproliferative agents in a controlled-release manner over a period of time. The present devices include implants for insertion periungually or subungually that comprise an antihyperproliferative agent. The present kits include those comprising a composition or device comprising an antihyperproliferative agent and instructions for inserting the composition periungually or subungually in a subject affected by a hyperproliferative disease. In one embodiment of the present invention, the hyperproliferative disorder is nail melanoma and the active ingredient is selected from rapamycin.

The present invention is useful for the treatment of microbial infections of the nail unit, such as but not limited to onychomycosis, as well as treatment of autoimmune disorders of the nail unit, such as but not limited to nail psoriasis, as well as treatment of hyperproliferative disorders of the nail unit, such as but not limited to nail melanoma.

In another aspect of the present invention is provided a composition therapy including the compositions and implants of the present invention.

In yet another aspect of the present invention is provided kits comprising a composition or implant of the present invention and instructions for depositing the composition or implant under or near the nail of a subject suffering from a disease of the nail unit.

In yet another aspect of the present invention is provided kits for the treatment of a disease of the nail unit in a subject suffering from the disease of the nail unit compositions and implants of the present invention.

In yet another aspect of the present invention is provided a use of a composition comprising a flowable composition in the manufacture of a medicament for the treatment of a disease of the nail unit.

In yet another aspect of the present invention is provided a use of a composition comprising (i) a controlled release subungual implant comprising a therapeutically effective active agent, or (ii) a flowable composition suitable for depositing subungually and comprising a therapeutically effective active agent suitable for subungually forming in situ a controlled release implant; in the in the treatment of a disease of the nail unit.

In yet another aspect of the present invention is provided a controlled release implant injection system for subungually depositing a controlled release flowable composition, the system comprising a syringe comprising a cartridge and an injection needle, wherein the needle is a 19 gauge needle or narrower, and the cartridge containing a flowable composition which may flow through the needle and which is suitable for subungually forming in situ a controlled release biodegradable implant system.

As used herein, “treatment” means any manner in which the symptoms of the disease are ameliorated or otherwise beneficially altered. Treatment also encompasses prophylaxis. For example, the present invention is useful for preventing relapse in patients who have previously been cured of the condition.

As used herein, “antifungal” means a compound or mixture of compounds that kills, destroys, inhibits, or inactivates a fungus.

As used herein, “antimicrobial” means a compound or mixture of compounds that kills, destroys, inhibits or inactivates a microbe, e.g., a fungus, a bacteria, a yeast, or a virus. An antimicrobial as used herein is meant to include antibiotic agents, antibacterial agents, antifungal agents, amongst others.

As used herein, “immunosuppressive” means a compound or mixture of compounds that inhibits the activity of the immune system.

As used herein, “antihyperproliferative” means a compound or mixture of compounds that inhibits the disregulation of cellular proliferation, for example, tumor cells.

As used herein, the term “subject” is not limited to a specific species or sample type. For example, the term “subject” may refer to a patient, and frequently a human patient. However, this term is not limited to humans and thus encompasses a variety of mammalian species.

As used herein, “disease of the nail unit” is meant to refer to conditions of the nail unit, or its surrounding tissue. Examples of such diseases include microbial infections such as fungal, bacterial and vital infections, hyperproliferative conditions such as cancers of the nail unit, and autoimmune diseases of the nail unit. Specific examples of diseases include onychomycosis, paronychia, nail psoriasis, nail melanoma, etc. Onychomycosis includes distal, proximal, superficial, white, black, or total dystrophic onychomycosis. Paronychia includes bacterial, fungal infections. Vital infections include nail warts (periungual and subungual), paravaccinia virus and herpes infection. Hyperproliferative disorders of the nail unit include tumors of the nail unit and include epithelial tumors, actinic keratinosis, basal cell carcinoma, soft tissue tumors such as fibrous tumors, vascular tumors, tumors of peripheral nerves, degenerative tumors and melanocytic lesions.

As used herein “therapeutically active agent” or “therapeutically active ingredient”, are used interchangably, as well as “active agent” and “active ingredient” which are used interchangeably, are meant to refer to therapeutic agents that ameliorate or prevent the disease state affecting the nail unit. These drugs include but are not limited to antimicrobials including antibiotics, antimycobacterials, antiamebics, antivirals, and antifungals and the like; antihyperproliferative agents including anti-cancer agents, anti-tumor agents, anti-neoplastic agents and the like; immunosuppressive agents include anti-psoriatic agents, steroidal and non-steroidal anti-inflammatory agents and the like. Many of these active agents can exist in different pharmaceutically acceptable salt forms. Likewise, many of the therapeutically active agents can exist as different polymorphs and isomers, as well as prodrugs, analytes thereof and metabolites thereof, and can be substituted with ease.

As used herein, the term “subungual” is meant to include the space under the nail plate, and including the space between the nail plate and the nail bed. The nail includes both finger and toe nails.

As used herein, the term “periungual” is meant to include to the area surrounding the nail, or involving the nail folds. The nail includes both finger and toe nails.

As used herein, “a” or “an” means “at least one” or “one or more.”

### DETAILED DESCRIPTION OF EMBODIMENTS

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The compositions of the present invention comprise an active ingredient and a pharmaceutically acceptable carrier material. The compositions herein must comprise an active ingredient when they are at the treatment site in or around the nail. However, the composition herein do not necessarily comprise active ingredient before they reach the treatment site. For example, the active ingredient and the carrier material can be injected simultaneously at the treatment site so that they combine in-situ.

Compositions of the present invention are intended for use for the treatment of a variety of diseases of the nail unit. When compositions of the present invention are to be used for the treatment of fungal infections of the nail unit, the compositions comprise any suitable antifungal active. Examples of antifungal actives include ciclopirox, naftifine, griseofulvin, itraconazole, terbinafine, ketoconazole, fluconazole, as well as rapamycin and FK506, and mixtures thereof. In certain embodiments of the present invention antifungals include ciclopirox, naftifine, terbinafine, and combinations thereof.

When compositions of the present invention are to be used for the treatment of bacterial infections of the nail unit, the compositions comprise any suitable antibacterial or antibiotic, which are used interchangeably herein. Examples of antibiotic actives include penicillins, cephalosporins, vancomycins, bacitracin, polymycins, tetracyclines, erythromycin, streptomycin and the like.

When compositions of the present invention are to be used for the treatment of antiviral infections of the nail unit, for example periungual warts, the compositions comprise any suitable antiviral. Examples of antiviral actives include, but are not limited to bleomycin, acyclovir, valcyclovir, famcyclovir, and the like.

When compositions of the present invention are to be used for the treatment of autoimmune disorders of the nail unit, the compositions comprise any suitable immunosuppressive agent. Examples of immunosuppressive agent include, but are not limited to, cyclosporin, rapamycin, tacrolimus, corticosteroids, FK506, mycophenolic acid, pimecrolimus, mutomonab-CD3, basiliximab, daclimuzab, azasan, efalizumab, and alefacept, and the like.

When compositions of the present invention are used for the treatment of hyperproliferative disorders of the nail unit, the compositions comprise any suitable antihyperproliferative agent. Examples of antihyperproliferative agents include, but are not limited to, rapamycin and the like, as well as anti-cancer agents, anti-tumor agents, anti-neoplastic agents, and the like.

Compositions of the present invention include flowable compositions suitable for periungually or subungually forming in situ a controlled release biodegradable implant system In a preferred embodiment, the composition comprises a biodegradable pharmaceutically acceptable thermoplastic polymer that is at least substantially insoluble in aqueous medium or body fluid, a pharmaceutically acceptable biocompatible solvent that is water soluble, and a therapeutically effective amount of an active ingredient, wherein the thermoplastic polymer and biocompatible solvent are present in concentrations effective to form the implant in situ. The active ingredient may be miscible in the polymer and/or solvent to provide a homogeneous mixture with the polymer, or insoluble to varying degrees in the polymer and/or solvent to form a suspension or dispersion with the polymer.

The present invention also provides implants comprising biologically active agents suitable for use subungually and periungually, and methods for producing the same, as well as methods of using the implants in the treatment of diseases of the nail unit These implants are solid articles and include microcapsules, microparticles, structured articles such as sutures, staples, medical devices, stents and the like as well as monolithic implants and implant films, filamentous membranes and matrices. It is preferred that the implant devices are biodegradable.

Suitable microcapsules are preferably dimensioned in the order of 10 to 400 microns, and preferably are dimensioned so as to avoid causing emboli if introduced into the blood stream of a mammal. They are typically composed of a porous shell of the thermoplastic, branched polymer and a core of another material such as a bioactive agent or a bioactive agent in a diluent or carrier. Various techniques for the preparation of microparticles are taught in the prior art.

Suitable microparticles have approximately the same dimensions as microcapsules. The microparticles are typically composed of a porous matrix of a thermoplastic polymer and bioactive agent. The bioactive agent is typically contained within the polymer matrix as a homogeneous dispersion or solution, or as heterogeneous domains. Suitable techniques for the preparation of microparticles are known in the prior art.

Suitable structured articles have the known shapes as indicated by the information conveyed by their names. The monolithic implants are single body implants formed outside the body by solidification of the flowable composition in an aqueous medium. The differing shapes may be obtained by use of a molding or extrusion device designed to provide such shapes as the flowable composition is contacted with the solidification bath. These implants preferably have shapes suitable for insertion under the nails of a subject suffering from a disease of the nail unit. Suitable structured articles may be prepared in accordance with well established techniques in the art.

Suitable films may be formed by casting upon the aqueous medium or by other techniques known to provide such films.

Suitable filamentous membranes may be formed by the technique of described in copending U.S. patent application Ser. No. 09/110,723, filed Jul. 7, 1998, the disclosure of which is incorporated herein by reference.

It is preferred that the compositions and implants herein are controlled-release compositions. That is, compositions that release an active ingredient over a period of time, for example, at least about 2 days, or at least about 7 days, or over a period of at least about 10 days, or at least about 14 days, or over one month or more. Preferred embodiments include those that release an active ingredient over 2 months or more, or three months or more. The composition of a suitable controlled-release composition may be tailored according to the release time required, for example the release period of the implanted composition being about 1 to about 6 months. In a preferred embodiment, the composition comprises a biodegradable polymer (poly-lactide co-glycolide) based delivery system and the polymer composition is selected to provide a release time of about 1 to about 6 months.

In embodiments where long term treatment is required, for example in onychomycosis where continuous dosing with an anti-fungal active may be necessary over extended periods of time, for example nine months or more, compositions and implants with a release period of one month, or two months, or three months, or six months, are prepared and used in the treatment repeatedly as required. Advantageously, the use of such extended release compositions can result in reduction in overall pain and discomfort to the subject during the treatment period, as well as increased patient compliance with treatment regimes. In an embodiment, the composition or implant is biodegradable thus obviating the need for surgical procedures to remove the composition or implant. In a preferred embodiment, the compositions and devices are implanted subdermally and sufficiently near the nail, or subungually, so as to afford accumulation of the active ingredient in and around the nail while at the same time minimizing systemic exposure.

The ability of the nail plate to store a drug is known in the art. Thus, in an embodiment of the present invention, compositions and implants of the present invention are deposited periodically that take advantage of the ongoing presence of active ingredient in the nail plate, even after release of the active ingredient from the implant is complete. Subsequent treatment with additional compositions and implants of the present invention may be timed to account for the ongoing presence of active ingredient in the nail plate.

In another embodiment of the present invention, the methods and compositions and implants of the present invention are used in combination with additional therapies for the treatment of diseases of the nail unit. In a preferred embodiment, a controlled release composition or implant of the present invention deposited subungually or periungually is used in combination with a topical treatment. Such combination treatments can be particularly advantageous when treatment of both the nail bed and nail plate are required, for example, in certain forms of onychomycosis. As an example, it is envisioned that the use of a topical lacquer containing terbinafine hydrochloride be combined with the use of a subungually deposited controlled release subungual or periungual implant of the present invention containing terbinafine hydrochloride for the treatment of onychomycosis. In other embodiments, it is envisioned that oral compositions for systemic delivery of an active ingredient be combined with compositions and implants of the present invention for the treatment of various diseases of the nail unit, including onychomycosis and nail psoriasis. In other embodiments, it is envisioned that suitable conventional therapies as previously discussed herein for the treatment of the various diseases of the nail unit be used in combination with the compositions and implants of the present invention.

In yet another embodiment of the present invention, the active ingredient used in the methods and compositions and devices of the present invention may comprise more than one active ingredient. For example, the active ingredient may comprise a combination of active ingredients that demonstrate an additive or synergistic effect. Methods to determine such combinations are well-known to those of skill in the art.

Preferred carrier materials for use herein are flowable compositions that are suitable for use as a controlled release implant for the active ingredient. The flowable compositions preferably include a biodegradable thermoplastic polymer that is at least substantially insoluble in an aqueous medium or body fluid. The thermoplastic polymers can be made from a variety of monomers which form polymer chains or monomeric units joined together by linking groups. These include polymers with polymer chains or backbones containing such linking groups as ester, amide, urethane, anhydride, carbonate, urea, esteramide, acetal, ketal, and orthocarbonate groups as well as any other organic functional group that can be hydrolyzed by enzymatic or hydrolytic reaction (i.e., is biodegradable by this hydrolytic action). These polymers are usually formed by reaction of starting monomers containing the reactant groups that will form these backbone linking groups. For example, alcohols and carboxylic acids will form ester linking groups. Isocyanates and amines or alcohols will respectively form urea or urethane linking groups. Preferably, the biodegradable thermoplastic polymer is selected from polyesters, polylactides, polyglycolides, polycaprolactones, polyanhydrides, polyamides, polyurethanes, polyesteramides, polydioxanones, polyacetals, polyketals, polycarbonates, polyorthocarbonates, polyorthoesters, polyphosphoesters, polyphosphazenes, polyhydroxybutyrates, polyhydroxyvalerates, polyalkylene oxalates, polyalkylene succinates, poly(malic acid), poly(amino acids), and copolymers, terpolymers, or combinations or mixtures of the above materials. More preferably, the biodegradable thermoplastic is a polyester. Preferably the polyester is a polylactide, a polyglycolide, a copolymer thereof, a terpolymer thereof, or a combination thereof.

In an embodiment, the flowable composition comprises an Atrigel® biodegradable polymer (poly-lactide co-glycolide) based delivery system, wherein the polymers are dissolved in a biocompatible solvent. In a preferred embodiment, flowable compositions are those described in U.S. Pat. Nos. 5,278,201; 5,324,519; and 6,395,293, the disclosures of which are incorporated herein by reference. In other embodiments, the polymers for use in the flowable compositions comprise biodegradable polymer (poly-lactide co-glycolide) based delivery systems, the blend ratio of monomers being generally about 90/10 to 10/90 (by weight) and preferably about 25/75 through about 75/25. In other embodiments, the biodegradable polymer is selected from 75/25 PLG; 85/15 PLG; 85/15 PLGH or 80/20 PLGH. Suitable biodegradable polymers for use in the compositions of the present invention are those that afford release of the specific active agent over the intended period of time in situ. Testing methods to select such suitable biodegradable polymers for use in the present invention with an active ingredient are well-known to persons of skill in the art, and include, but are not limited to, methods as set out in the Examples as described herein.

The preferred biocompatible thermoplastic polymers for use herein have a lower degree of crystallization and are more hydrophobic. Preferably, the thermoplastic polymer is substantially soluble in the organic solvents so that up to 50-60 wt % solids can be made. Preferably, the polymers used according to the invention are essentially completely soluble in the organic solvent so that mixtures up to 85-98 wt % solids can be made. Preferably the polymers are at least substantially insoluble in water so that less than 0.1 g of polymer per mL of water will dissolve or disperse in water. Preferably, the polymers used according to the invention are essentially completely insoluble in water so that less than 0.001 g of polymer per mL of water will dissolve or disperse in water.

Solvents suitable for use in the flowable composition are biocompatible and are at least slightly soluble in aqueous medium, body fluid, or water. The organic solvent preferably is at least moderately soluble, more preferably very soluble, and most preferably soluble at all concentrations in aqueous medium, body fluid, or water. Preferably, the organic solvent has a molecular weight in the range of about 30 to about 1000.

Examples of biocompatible organic solvents that may be used to form the flowable compositions of the present invention include aliphatic, aryl, and arylalkyl linear, cyclic and branched organic compounds that are liquid or at least flowable at ambient and physiological temperature and contain such functional groups as alcohols, ketones, ethers, amides, esters, carbonates, sulfoxides, sulfones, and any other functional group that is compatible with living tissue.

Preferred biocompatible organic solvents that are at least slightly soluble in aqueous or body fluid include N-methyl-2-pyrrolidone, 2-pyrrolidone; C1 to C15 alcohols, diols, triols and tetraols such as ethanol, glycerine, propylene glycol, butanol; C3 to C15 alkyl ketones such as acetone, diethyl ketone and methyl ethyl ketone; C3 to C15 esters such as methyl acetate, ethyl acetate, ethyl lactate; C1 to C15 amides such as dimethylformamide, dimethylacetamide and caprolactam; C3 to C20 ethers such as tetrahydrofuran, or solketal; tweens, triacetin, propylene carbonate, decylmethylsulfoxide, dimethyl sulfoxide, oleic acid, and 1-dodecylazacycloheptan-2-one. Other preferred solvents are benzyl alcohol, benzyl benzoate, dipropylene glycol, tributyrin, ethyl oleate, glycerin, glycofural, isopropyl myristate, isopropyl palmitate, oleic acid, polyethylene glycol, propylene carbonate, and triethyl citrate. Preferred solvents include N-methyl-2-pyrrolidone, 2-pyrrolidone, dimethyl sulfoxide, triacetin, and propylene carbonate. Biocompatible organic solvents that may be used also include combination of biocompatible solvents, including combination of the biocompatible solvents as described herein.

Preferably the concentration of the polymer in the organic solvent according to the invention will range from about 0.01 g per ml of solvent to a saturated concentration. Typically, the saturated concentration will be in the range of 80 to 95 wt % solids or 4 to almost 5 gm per ml of solvent assuming that the solvent weighs approximately 1 gm per ml.

Any suitable concentration of active ingredient may be used, where the active ingredient is administered in an effective amount to achieve its intended purpose. Determination of a therapeutically effective amount for a particular active ingredient is well within the capability of persons skilled in the art, especially in light of the detailed disclosure provided herein. Moreover, it is further preferred that the selection of an active agent when used with compositions of the present invention containing thermoplastic polymers is made such that the active ingredient does not contain functional groups that will not interfere with the polymers used in the flowable formulations. In one embodiment, the active is preferably present in about 0.1 wt. % to about 50 wt. %. In other embodiments, the active is preferably present in about 0.1 wt. % to about 30 wt. %, more preferably from about 1 wt. % to about 30 wt. %, even more preferably from about 5 wt. % to about 25 wt. %, of the composition. In embodiments using polylactide, a polyglycolide, a copolymer thereof, a terpolymer thereof, or a combination thereof as a thermoplastic polymer, preferably the active ingredient is present in about up to 30 wt. % of the composition. In other embodiments, the preferred concentration of active utilized is that which maximizes the amount of active ingredient present in a minimal volume of composition or implant, without adversely impacting localized and/or systemic toxicity. In other embodiments, the ranges preferred are up to about 5 wt %, or up to about 4 wt % or up to about 3 wt % or up to about 2 wt % or up to about 1 wt %. In one embodiment, the flowable composition comprises tetbinafine hydrochloride as the active at about 1 wt %. In another embodiment, the flowable composition comprises terbinafine hydrochloride as the active at less than 5 wt. %.

Preferably, the flowable composition is formulated as an injectable composition. Preferably, the flowable composition is injected subungually or periungually. The injectable composition preferably has a volume of about 0.01 mL to about 1.0 mL or about 0.02 mL to about 1.0 mL, or about 0.05 mL to about 0.5 mL. Preferably, the flowable composition is a liquid or a gel composition, suitable for injection subungually or periungually in a subject. In a preferred embodiment, the injectable composition has a volume that minimizes the size of the implant formed in situ under the nail.

Preferably the flowable composition is suitable for injection under the nails of a subject where it forms a pharmaceutically acceptable, solid matrix. In one aspect of the flowable composition, a biologically active agent is included and the solid implant will release the active agent at a controlled rate. The rate of release may be altered to be faster or slower by inclusion of a rate-modifying agent that are well known in the art.

Preferably, the composition or implant is in the form of a flowable composition, solution or suspension which is injected with a needle having a gauge of 19 or higher, more preferably 20 or higher, or 25 or higher. The gauge of the needle is selected such that the flowable composition may flow through the needle into the subungual or periungual space, while at the same time minimize local trauma and discomfort in the subject. In a preferred embodiment, the flowable composition, for example those as described herein comprising a biodegradable pharmaceutically acceptable thermoplastic polymer and a biocompatible solvent, remains substantially deposited subungually or periungually upon withdrawal of the needle. In a preferred embodiment, the composition to be injected comprises a flowable gel, or a suspension or a solution, and the composition is injected with a small gauge needle, preferably 19 or higher, more preferably 20 or higher, or 25 or higher.

In other embodiments of the invention are provided methods of treating a disease of the nail unit, including antimicrobial infections of the nail unit, hyperproliferative disorders of the nail unit and autoimmune disorders of the nail unit. Such methods include, but are not limited to, treating onychomycosis, nail psoriasis and nail melanoma. In one embodiment, methods comprise insetting a composition or implant as described herein subungually or periungually in a subject. Such an insertion is preferably in the form of an injection. Preferably the method comprises inserting a flowable composition as described hereinabove subungually or periungually in a subject. In an embodiment, a flowable composition is inserted subungually through an opening preformed in the nail, to afford placement of the flowable composition through the nail plate to the subungual space. Preferably, flowable compositions are formulated so as to minimize the injection volume of flowable composition and therefore the implant size, while at the same time are formulated to contain the desired therapeutically effective amount of active agent for prolonged delivery over a desired period of time. In another embodiment, the flowable compositions are capable of forming in situ a controlled release implant that advantageously adopts a shape subungually or periungually that conforms to the subungual or periungual space available. It is believed that this may lead to a decrease in undesired overt or localized tissue trauma experienced upon deposition of the implant into the subungual or periungual space. In another embodiment, upon deposition/injection and contact with tissue fluid, the composition progressively solidifies over a period of time, preferably hours. During this period, the shape of the implant is manipulated, for example by palpitation, and the volume of the implant expands.

When a subject is suffering from a disease of the nail unit that results in separation of the nail plate from the nail bed, such as in certain forms of onychomycosis, insufficient tissue fluid may be present to afford formation of the flowable composition into a solid matrix. In such cases, the flowable composition is deposited topically in the subungual space between the nail plate and nail bed and a suitable amount of aqueous solution is introduced in a suitable manner to the composition, either simultaneously or sequentially, so as to afford formation of the solid matrix implant. In one embodiment, the additional source of aqueous solution may be provided by bathing or showering the nail with water or otherwise introducing water to the flowable composition under the nail, so as to provide sufficient water for implant formation subungually.

The present invention includes kits comprising a composition or implant as described hereinabove and instructions for use of such compositions or implants. In one embodiment, the composition or implant includes an antifungal active and instructions preferably comprise a description of inserting the composition or implant subungually or periungually in an onychomycosis sufferer. In another embodiment, the composition or implant includes an immunosuppressive active and instructions preferably comprise a description of inserting the composition or implant subungually or periungually in a nail psoriasis sufferer. In yet another embodiment, the composition or implant includes an antihyperproliferative active and instructions preferably comprise a description of inserting the composition or implant subungually or periungually in a nail melanoma sufferer. Preferably the kit comprises a biodegradable thermoplastic polymer, a suitable active ingredient as described herein, and instructions for injecting the polymer and active subungually or periungually in a subject.

In a further embodiment the kit comprises first container comprises a composition comprising a biodegradable thermoplastic polyester that is at least substantially insoluble in aqueous medium or body fluid and a biocompatible polar aprotic solvent selected from an amide, an ester, a carbonate, a ketone, an ether, and a sulfonyl; wherein the biocompatible polar aprotic solvent is miscible to dispersible in aqueous medium or body fluid; and a second container comprising an antifungal active. Preferably the containers are syringes. Preferably the kit comprises instructions. Preferably the first container can be connected to the second container.

It will be understood that the following embodiments of the present invention are intended to be illustrative of some of the possible applications or principles. Various modifications may be made by the skilled person without departing from the true spirit and scope of the invention.

### Example 1

### Preparation of Polymers

A 360 ml Teflon vessel was charged with D,L lactide (275 g), polyol (0.4-1.1 w/w %), and stannous octoate (0.045 w/w %). The mixture was heated at 145° C. for 20 hours. The resulting polyester was removed from the reaction vessel and dissolved in anhydrous dichloromethane and purified by precipitation in anhydrous methanol. The polymers were dried under vacuum at ambient temperature to remove most of the residual solvent. The resulting hard, solid masses were cooled in liquid nitrogen and cut into small pieces. The small pieces were ground in a Wiley mill to a coarse dust sufficient to pass through a 6 mm screen. The resulting polymer was dried under vacuum at ambient temperature prior to final packaging.

### Example 2

### Local Injection for Onychomycosis

The biodegradable polymer of Example 1 is dissolved in N-methyl pyrollidone and sterilized. It is loaded into a 1 ml polypropylene syringe female luer lock fitting. Sterile terbinafine hydrochloride solution (1%) is loaded into another 1 ml syringe polypropylene syringe with a male luer-lock fitting. The syringes are coupled together and mixed until a homogenous mixture is formed. The formulation is drawn back into the syringe with the male coupling, the two syringes separated, and a one-half inch 20 gauge needle is attached. The contents of the syringe is injected under the nail of a 30 year old man suffering from onychomycosis. The efficacy of the implant in controlling onychomycosis symptoms is measured.

### Example 3

### Periungual Injection containing Terbinafine HCl for Onychomycosis

A biodegradable polymer (poly-lactide co-glycolide) based delivery system based implant composition for injection containing tetbinafine hydrochloride (1 wt %) and providing release of terbinafine hydrochloride over about a three month period is prepared as describe herein and prepared for injection as set forth in Example 2. One or more periungual injections of a small volume (25-50 μL) of the flowable compositions are made in a subject suffering from onychomycosis, following local anesthesia at the one or more injection sites. The efficacy of the implant in controlling onychomycosis symptoms is measured and the treatment is repeated as requited.

### Example 4

### Sublingual Implant containing Terbinafine HCl for Onychomycosis

Similar to Example 3, a based biodegradable polymer implant composition containing terbinafine hydrochloride and providing release of terbinafine hydrochloride over about a three month period in situ as an implant is prepared as describe herein and prepared for injection as set forth in Example 2. The composition is applied topically (non-invasively) under the loose end of a nail in patient having a nail plate that is separated from or loosely attached to the nail bed. The treated nail is then exposed to water to facilitate polymerization of the implant. The efficacy of the implant in controlling onychomycosis symptoms is measured and the treatment is repeated as required.

### Example 5

### Local injection for Nail Psoriasis using Rapamycin

A flowable composition for the in situ formation of a biodegradable implant containing rapamycin for the treatment of nail psoriasis is prepared as set following the procedure of Example 3. Sterile rapamycin solution (1%) is utilized in place of the terbinafine hydrochloride solution. One or more periungual injections of 25-50 μl is administered proximal to the affected nail(s) of a subject suffering from nail psoriasis, following local anesthesia at the injection sites. The efficacy of the implant in controlling nail psoriasis is determined. Utilizing compositions prepared to provide 3-6 month sustained release of rapamycin, the treatment is repeated every 3 to 6 months as needed_{[AI]}.

### Example 6

### Local injection for Nail Psoriasis Using Cyclosporin A

Following the Procedure in Example 5, a flowable composition for the in situ formation of a biodegradable implant containing cyclosporin A for the treatment of nail psoriasis is prepared and administered. Sterile cyclosporin A (1%) solution is utilized in place of the rapamycin solution. The efficacy of the implant in controlling nail psoriasis is determined. The treatment is repeated as required. Utilizing compositions prepared to provide 3-6 month sustained release of cyclosporin, the treatment is repeated every 3 to 6 months as needed.

### Example 7

### Local Injection for Nail Melanoma

A flowable composition containing rapamycin for the treatment of nail melanoma is prepared and utilized as set following the procedure of Example 5. The efficacy of the implant in controlling nail melanoma is measured. The treatment is repeated as required.

### Example 8

### Animal Implant Retrieval Studies with Terbinafine Hydrochloride

A 28 day study was carried out using a flowable composition of the present invention comprising a suspension formulation of 1 wt % terbinafine hydrochloride in 40 wt % 85/15 PLGH in NMP. A 100 μl quantity of the solution was injected subcutaneously into rats (n=5). The implants were retrieved at 1, 7, 14 and 28 days and the percent release of active ingredient of the implant compared (determined via solvent extraction of the remaining active from the implant and measured via HPLC) with the theoretically calculated 3 month release kinetics. Based upon the recovered active ingredient, the rate of release over the first 28 days was observed to be consistent with the theoretical three month release kinetics.

### Example 9

### Animal Implant Retrieval Studies with Terbinafine Hydrochloride

Similar to Example 8, 24 hour animal implant retrieval studies were carried out with 100 μL injections (solutions and suspensions) of flowable composition injected subcutaneously into rats (n=5 for each formulation). Flowable compositions (1% or 5% terbinafine hydrochloride with the following polymer systems in NMP: 30% 50/50 PLGH/20% 85/15 PLGHp; 40% 85/15 PLGHp; 45% 75/25 PLG in NMP; 50% 85/15 PLG; 45% 75/25 PLG; 55% 75/25 PLG; 40% 75/25 PLGH). The implants were removed after 24 hours and the percentage of active ingredient recovered in the implant determined by HPLC. All formulations showed release of the active ingredient from the implant to varying degrees. Release rated varied for different formulations tested and several tested formulations were identified with greater than 85% retention of terbinafine hydrochloride in the implant.

### Example 10

### Tissue Studies with Terbinafine Hydrochloride

Tissue studies using various the compositions as described in Examples 8 and 9 were carried out in rats. The formulations comprising terbinafine hydrochloride (1 wt % or 5 wt %) were prepared as described herein and injected at a subcutaneous volume of 25 or 100 μl. Injections were made with a 19 gauge thin-walled ⅝ inch length needle. Body weights of animals were collected prior to treatment. Animals were anesthetized for injection. A single, 100 μl (approximate) subcutaneous injection was administered in the dorsal thoracic region. Animals were monitored post-administration for signs of overt systemic or localized (injection site) toxicity and the observations were recorded. Animals were deeply anesthetized and blood was collected by cardiac puncture. The animals were euthanized and gross necropsies were performed on one animal per group. Localized areas of injection sites were examined on all animals and observations recorded (vasodilation, erythema, edema was evaluated). Implants were retrieved. Injection site and implant characteristics were recorded. It was noted that implants containing 1% terbinafine hydrochloride did not generally cause local effects to the surrounding tissue; some internal edema and encapsulation was noted for implants tested containing 5% terbinafine hydrochloride.

### Example 11

### Implant Retrieval Studies with Cyclosporin A

Similar to Example 9, 24 hour animal implant retrieval studies (n=5 for each formulation) were carried out using flowable compositions containing 1% cyclosporin A with the following in NMP: 45% 75/25 PLG in NMP; 50% 85/15 PLG; 45% 75/25 PLG; 55% 75/25 PLG; 40% 75/25 PLGH; 40% 85/15 PLGH. Briefly, test formulations were prepared by weighing the Atrigel formulation into a female syringe with stopper. Atrigel formulations were prepared by mixing polymer and NMP in vials, and the polymer dissolved by spinning until dissolved. The active ingredient powder was weighed into a male syringe with stopper. Prior to use, both syringes were coupled and mixed for 100 cycles until the formulation was homogenized. One mixing cycle was considered to be the pushing of the contents of both syringes back and forth. The implants were removed after 24 hours and the percentage of active ingredient recovered in the implant determined by HPLC. Formulations showed release of the active ingredient from the implant to varying degrees, from a negligible level of release to up to about 6% release.

### Example 12

### Tissue Studies with Cyclosporin A

Similar to Example 10, tissue studies were conducted with implants containing 1 wt % cyclosporin A with the following in NMP: 55% 75/25 PLG; 40% 75/25 PLGH; 50% 85/15 PLG (n=5 for each formulation). All implants tested containing cyclosporin A were well tolerated in rats following subcutaneous injection.

Analysis of the implants as in Example 8 above, for 28 day release kinetics revealed release of active ingredient from the implants, with varying degrees for each formulation.

### Example 13

### Syringability Studies

Solution and suspension formulations of 1% tetbinafine hydrochloride in 40% 85/15 PLGH in NMP were injected through 25 G needles into clean glass vials. Qualitative observations were taken on the difficulty of syringeability. The same formulations were injected without using needles into clean glass vials. The samples were dissolved and analyzed by HPLC for terbinafine hydrochloride content.

The formulations were found to be syringeable without requiring excessive force to pass through the needle. Percent recoveries of suspension and solution formulations were 95.2±2.7% and 99.2±1.1%, respectively.

The ordinarily skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, not does it constitute any admission as to the contents or date of these publications or documents.

1. A flowable composition suitable for subungually or periungually forming in situ a controlled release implant system, the composition comprising:
a. a biodegradable pharmaceutically acceptable thermoplastic polymer that is at least substantially insoluble in aqueous medium or body fluid;
b. a pharmaceutically acceptable biocompatible solvent that has a solubility in water from essentially insoluble to soluble in all proportions; and
c. a therapeutically effective amount of an active agent;
wherein the thermoplastic polymer and biocompatible solvent are present in concentrations effective to form the implant in situ subungually or periungually.

2. The flowable composition of claim 1, wherein:
a. the biodegradable pharmaceutically acceptable thermoplastic polymer comprises a biodegradable thermoplastic polyester that is at least substantially insoluble in aqueous medium or body fluid; and
b. the pharmaceutically acceptable biocompatible solvent comprises biocompatible polar aprotic solvent selected from the group consisting of an amide, an ester, a carbonate, a ketone, an ether, and a sulfonyl; wherein the biocompatible polar aprotic solvent is miscible to dispersible in aqueous medium or body fluid.

3. The composition of claim 2 wherein the biodegradable thermoplastic polyester comprises a polylactide, a polyglycolide, a polycaprolactone, a copolymer thereof, a terpolymer thereof, or any combination thereof.

4. The composition of claim 4 wherein the biodegradable thermoplastic polyester comprises a polylactide, a polyglycolide, a copolymer thereof, a terpolymer thereof, or a combination thereof.

5. The composition of claim 2 wherein the biocompatible polar aprotic solvent comprises N-methyl-2-pyrrolidone, 2-pyrrolidone, N,N-dimethylformamide, dimethyl sulfoxide, propylene carbonate, caprolactam, triacetin, or any combination thereof.

6. The flowable composition of claim 1, wherein the active ingredient comprises an antimicrobial agent, an immunosuppressive agent, or an antihyperproliferative agent.

7. The flowable composition of claim 1, wherein the active agent comprises an antifungal agent.

8. The composition of claim 7, wherein the antifungal agent is selected from ciclopirox, naftifine, griseofulvin, itraconazole, terbinafine, ketoconazole, fluconazole, rapamycin and FK506, mixtures thereof, and suitable salts or derivatives thereof.

9. The flowable composition of claim 1, wherein the active agent comprises an antiviral agent.

10. The flowable composition of claim 9, wherein the antiviral agent is selected from bleomycin, acyclovir, valcyclovir, and famcyclovir, suitable salts and derivatives thereof, and mixtures thereof.

11. The flowable composition of claim 1, wherein the active agent comprises an antibiotic agent.

12. The flowable composition of claim 11, wherein the active agent is selected from penicillins, cephalosporins, vancomycins, bacitracin, polymycins, tetracyclines, erythromycin, streptomycin, suitable salts and derivatives thereof, and mixtures thereof.

13. The flowable composition of claim 1, wherein the active agent comprises an immunosuppressive agent.

14. The composition of claim 13, wherein the immunosuppressive agent is selected from cyclosporin, rapamycin, tacrolimus, corticosteroids, FK506, mycophenolic acid, pimecrolimus, muromonab-CD3, basiliximab, daclimuzab, azasan, efalizumab, and alefacept, suitable salts and derivatives thereof, and mixtures thereof.

15. The flowable composition of claim 1, wherein the active agent comprises an antihyperproliferative agent.

16. The composition of claim 15, wherein the antihyperproliferative agent is selected from rapamycin.

17. (canceled)

18. A combination therapy for use in the treatment of a disease or disorder of the nail unit selected from a microbial infection, an autoimmune disease and a hyperproliferative disease, the combination therapy comprising a composition of claim 1 used in combination with a second therapy.

19. The combination therapy of claim 18, wherein the second therapy comprises a topical therapy or an oral therapy.

20. A method for the treatment of a disease of a nail unit, the method comprising depositing a composition comprising:
(i) a controlled release implant suitable for depositing subungually and comprising a therapeutically effective active agent, or
(ii) a flowable composition comprising an therapeutically effective active agent suitable for subungually forming in situ a controlled release implant;
under the nail of a subject suffering from the disease of the nail unit.

21. The method of claim 20, wherein the flowable composition or implant comprises a biodegradable implant.

22. The method of claim 21, wherein the flowable composition comprises a biodegradable thermoplastic polyester that is at least substantially insoluble in aqueous medium or body fluid.

23. The method of claim 20, where the flowable composition is according to claim 1.

24. The method of claim 20, where the implant is in the form of microcapsules, microparticles, sutures, staples, medical devices, stents, monolithic implants, implant films, filamentous membranes or matrices.

25. The method of claim 20, wherein the disease of the nail unit comprises a microbial infection and the active ingredient comprises a therapeutically effective antimicrobial agent.

26. The method of claim 25, wherein a composition comprising a therapeutically effective amount of an antifungal active is deposited under the nail of the subject to treat onychomycosis in a subject suffering therefrom.

27. The method of claim 26, wherein the antifungal is selected from ciclopirox, naftifine, griseofulvin, itraconazole, terbinafine, ketoconazole, fluconazole, rapamycin and FK506, salts and derivatives thereof, and mixtures thereof.

28. The method of claim 26, wherein the treating comprises at least one of killing or decreasing the growth of a pathogenic fungus infecting the nail, and improving the clinical abnormality in nail appearance.

29. The method of claim 25, wherein a composition comprising a therapeutically effective amount of an antiviral active is deposited under the nail of the subject to treat a viral infection of the nail unit in a subject suffering therefrom.

30. The method of claim 29, wherein the antiviral is selected from bleomycin, acyclovir, valcyclovir, and famcyclovir, salts and derivatives thereof, and mixtures thereof.

31. The method of claim 25, wherein a composition comprising a therapeutically effective amount of an antibacterial active is deposited under the nail of the subject to treat a bacterial infection of the nail unit in a subject suffering therefrom.

32. The method of claim 31, wherein the antibiotic is selected from penicillins, cephalosporins, vancomycins, bacitracin, polymycins, tetracyclines, erythromycin, streptomycin, salts and derivatives thereof, and mixtures thereof.

33. The method of claim 20, wherein the disease of the nail unit comprises a hyperproliferative disease and the active ingredient comprises a therapeutically effective antihyperproliferative agent.

34. The method of claim 33, wherein a composition comprising a therapeutically effective amount of an antihyperproliferative active is deposited under the nail of the subject to treat nail melanoma in a subject suffering therefrom.

35. The method of claim 34, wherein the antihyperproliferative agent is selected from rapamycin, or derivatives thereof.

36. The method of claim 20, wherein the disease of the nail unit comprises an autoimmune disease and the active ingredient comprises a therapeutically effective immunosuppressive agent.

37. The method of claim 36, wherein a composition comprising a therapeutically effective amount of an immunosuppressive agent is deposited under the nail of the subject to treat nail psoriasis in a subject suffering therefrom.

38. The method of claim 37, wherein the immunosuppressive agent is selected from cyclosporin, rapamycin, tacrolimus, corticosteroids, FK506, mycophenolic acid, pimecrolimus, muromonab-CD3, basiliximab, daclimuzab, azasan, efalizumab, and alefacept, suitable salts and derivatives thereof, and mixtures thereof.

39. The method of claim 20, wherein the composition comprises a flowable composition and is deposited via injection.

40. The method of claim 39, wherein the composition is deposited subungually via injection between the nail bed and the nail plate via entry through the hyponychium.

41. The method of claim 39, wherein the composition is deposited subungually via injection through the nail plate.

42. The method of claim 41, wherein a passage is preformed in the nail plate and said composition is subsequently deposited subungually via injection through the passage in the nail plate.

43. The method of claim 39, wherein the composition is injection periungually.

44. A kit comprising a composition according to claim 1 and instructions for depositing the composition under the nail of a subject suffering from a disease of the nail unit.

45. A kit for the treatment of a disease of the nail unit in a subject suffering from the disease of the nail unit comprising:
(a) a first container comprising a composition comprising a biodegradable thermoplastic polyester that is at least substantially insoluble in aqueous medium or body fluid and a biocompatible polar aprotic solvent selected from an amide, an ester, a carbonate, a ketone, an ether, and a sulfonyl, wherein the biocompatible polar aprotic solvent is miscible to dispersible in aqueous medium or body fluid; and
(b) a second container comprising an active ingredient; and
(c) instructions.

46. An implant for use as a medicament for the treatment of a disease of the nail unit comprising a solid or gel composition produced from a flowable composition comprising a therapeutically effective active agent and a carrier wherein said carrier is suitable for subungually forming in situ a controlled release implant containing the active agent.

47. The implant of claim 46, wherein the flowable composition is a composition of claim 1.

48. An implant for use in the treatment of a disease of a nail unit, comprising a controlled release implant comprising a therapeutically effective active agent for treatment of the disease of the nail unit and a biodegradable pharmaceutically acceptable thermoplastic polymer that is at least substantially insoluble in aqueous medium or body fluid.

49. The implant of claim 48, produced from the composition of claim 1.

50. A controlled release implant injection system for subungually depositing a controlled release composition, the system comprising a syringe comprising a cartridge and an injection needle, wherein the needle is a 19 gauge needle or higher, and the cartridge containing a flowable composition which may flow through the needle and which is suitable for subungually forming in situ a controlled release biodegradable implant system.

51. The controlled release implant injection system of claim 50, wherein the needle is selected from a 19 gauge needle, a 20 gauge needle, a 25 gauge needle, or greater than a 25 gauge needle.

52. (canceled)

53. The implant of claim 49 wherein a portion of the organic solvent remains with the implant.

54. The implant of claim 50 wherein the organic solvent remaining with the implant eventually dissipates into body tissue.

55. The implant of claim 50 wherein the organic solvent remaining with the implant remains as long as the implant releases active agent.

56. The implant of claim 48 formed either ex vivo or in situ.

57. The implant of claim 48 present in the nail unit subungually or periungually.

58. The flowable composition of claim 2, wherein the active agent comprises an antifungal agent.

59. The composition of claim 53, wherein the antifungal agent is selected from ciclopirox, naftifine, griseofulvin, itraconazole, terbinafine, ketoconazole, fluconazole, rapamycin and FK506, mixtures thereof, and suitable salts or derivatives thereof.

60. The flowable composition of claim 2, wherein the active agent comprises an antiviral agent.

61. The flowable composition of claim 55, wherein the antiviral agent is selected from bleomycin, acyclovir, valcyclovir, and famcyclovir, suitable salts and derivatives thereof, and mixtures thereof.

62. The flowable composition of claim 2, wherein the active agent comprises an antibiotic agent.

63. The flowable composition of claim 57, wherein the active agent is selected from penicillins, cephalosporins, vancomycins, bacitracin, polymycins, tetracyclines, erythromycin, streptomycin, suitable salts and derivatives thereof, and mixtures thereof.

64. The flowable composition of claim 2, wherein the active agent comprises an immunosuppressive agent.

65. The composition of claim 59, wherein the immunosuppressive agent is selected from cyclosporin, rapamycin, tacrolimus, corticosteroids, FK506, mycophenolic acid, pimecrolimus, muromonab-CD3, basiliximab, daclimuzab, azasan, efalizumab, and alefacept, suitable salts and derivatives thereof, and mixtures thereof.

66. The flowable composition of claim 2, wherein the active agent comprises an antihyperproliferative agent.

67. The composition of claim 61, wherein the antihyperproliferative agent is selected from rapamycin.

68. A kit comprising a composition according to claim 2 and instructions for depositing the composition under the nail of a subject suffering from a disease of the nail unit.
